(19) United States
(12) Patent Application Publication
(10) Pub. No.: US 2019/0211299 A1
(43) Pub. Date: Jul. 11, 2019

(54) END FITTING AND DEVICE FOR SAMPLING COLONIES OF MICROORGANISMS AND SAMPLING PROCESS USING SAME

(71) Applicant: BIOMÉRIEUX, Marcy l'Etoile (FR)

(72) Inventors: Corinne BEAULIEU, Lachassagne (FR); Jean-Philippe CHARRIER, Tassin la Demi-Lune (FR); Frédéric FOUCAULT, Marcy l'Etoile (FR); Cécile PARIS, Bessenay (FR); Philippe WANDELS, Lyon (FR); Bouchra MAKROUF, Lyon (FR); Bruno COLIN, Marcy l'Etoile (FR)

(73) Assignee: BIOMÉRIEUX, Marcy l'Etoile (FR)

(21) Appl. No.: 16/310,782

(22) PCT Filed: Jun. 14, 2017
(86) PCT No.: PCT/EP2017/064633

§ 371 Date: Dec. 17, 2018

(30) Foreign Application Priority Data
Jun. 16, 2016 (EP) 16174789.4
Oct. 4, 2016 (EP) 16192227.3

(51) Int. Cl. C12M 1/30; B01L 3/02; G01N 1/02

(52) U.S. Cl. CPC C12M 33/02; G01N 1/02; B01L 3/0279; B01L 3/0275

(57) ABSTRACT

An end fitting capable of being fitted to the body of a manual or automated device for sampling a biological substance of microbial origin, including: a) a distal end including a means for sampling the biological substance of microbial origin, b) a proximal free end intended to come into contact with the body of the sampling device and to enable the attachment of the end fitting to said body, the end fitting being characterized in that all or some of the free distal end consists of a fibrous material having a porosity at least equal to 30%.

The technical field of the present invention is that of devices intended to sample colonies of microorganisms on a gelose culture medium for analysis. The present invention more particularly concerns a sampling end fitting having in its distal part a polymer material sampling tip. The invention also concerns a sampling device including an end fitting of this kind and a sampling process using same.

At present, a colony of microorganisms (bacteria, mold, yeast or the like) cultivated on a gelose culture medium in a Petri dish, or on any other medium, is sampled using tools that can be sterilized or single-use tools such as oeses (also known as loops), sticks, tubes or cones.

However, these tools do not enable reliable and efficient sampling of all types of microorganisms because the latter can have very diverse shapes, sizes, consistencies, structures or appearances.

Moreover, these consumables do not easily enable optimum deposition of the sampled biological substance on analysis media such as plates intended for MALDI-TOF type mass spectrometry analysis. Also, it is also very important to be able to sample a colony of bacteria or a fraction of that sample without sampling the culture medium under the colony. This can in fact falsify the results of subsequent analyses.

The quality of the results of analysis may also depend on the concentration of the deposit of the biological substance, formed from the sample taken, and its homogeneity on the medium on which it is deposited. This is particularly the case for MALDI-TOF microorganism analyses, in which the sample must form a thin and uniform layer to enable optimum analysis.

Oeses that are disposable or can be sterilized by flame are conventionally used to sample a colony of microorganisms on a Petri dish and to deposit the biological substance on the MALDI-TOF plate. This operation is not easy and calls for some dexterity. Gripping a tool of this kind between the thumb and the index finger can cause muscular-skeletal problems. The oese is generally held far from its end so that the hand of the operative does not contaminate the sample, but this position of the hand makes the accuracy of the movement at the end of the oese more difficult, notably when it is necessary to produce a thin and homogeneous deposit over a small area of the order of a few mm². Finally, the oese, having been designed to sample a calibrated given quantity of microorganisms (generally between 1 μL and 10 μL), includes at its end a metal or plastic loop which has a diameter that is generally greater than 1 mm. This tip can be larger than the surface on which the deposit is to be formed. Moreover, the rigid nature of the (metal or plastic) oese is not particularly suitable for spreading a colony of microbes in a thin uniform layer on a hard surface.

It is also possible to use other consumables such as swabs, wooden sticks, micropipette end fittings.

Thus, the document U.S. Pat. No. 9,181,522 describes a process and an apparatus for the aseptic transfer of biological substances. The apparatus is composed of a double-walled chamber for housing end fittings of a single size having a ball at the head end, intended for the transfer of the biological substance from one place to another, in an aseptic manner. A first disadvantage of a device of this kind is that is of relatively complex design, with an integral system for UV sterilization of the end fittings, its double-wall architecture and its system for internal loading and ejection of the end fittings. Such complexity undoubtedly impacts the unit cost and therefore the selling price. Moreover, the material used to make the ball present at the head end of the disposable end fitting is made of a hard material, of metal or polypropylene type, which is not propitious for the sampling process but above all for depositing a biological substance, such as a colony of bacteria, notably on a MALDI-TOF type mass spectrometry analysis plate.

The document FR 2 668 495 describes a sterile sampling cone for bacteriological use. Said cone has at its distal end a solid protuberance that is slightly frustoconical, off-axis relative to the longitudinal axis of the cone. This protuberance enables the sampling of a biological substance. To this end, in one particular embodiment, it may include an optional loop. Even with a particular architecture, the cone described in the above document is nevertheless still made of a material conventionally used for this kind of product. Namely a hard and smooth plastic material that is not suited to sampling and depositing biological substances. Moreover, its particular shape does not make it easy to use to deposit a biological substance, such as a colony of bacteria over a very small area, such as a MALDI-TOF mass spectrometry analysis plate.

The applicant has previously resolved all or some of the disadvantages referred to above by proposing a process for sampling all or some of a sample of a biological substance grown in contact with a gelose culture medium, which uses a probe provided with a terminal end. Said sampling process is essentially based on cooling the terminal end of the probe, enabling sticking of all or part of the sample of the biological substance to be sampled by contact of the terminal end with the sample of the biological substance or by application of a pressure exerted by the terminal end on the sample of the biological substance, followed by releasing all or some of the sample of the biological substance by heating the terminal end of the probe. This process is described in the patent application WO 2012/004545.

The main disadvantage of this process is that it necessitates relatively bulky equipment for cooling the probe, which consumes energy and represents a high financial cost.

The result of the analysis of the prior art is that at present there exists no system for sampling a biological substance that is easy to use, of simple design and employs a disposable sampling end fitting having physical properties suited to optimizing not only sampling but also depositing a biological substance such as a colony of bacteria.

The objectives of the present invention are therefore to address these lacks by proposing an end fitting that is of simple design, easy to produce, enables when placed on a device for sampling a biological substance precise sampling and deposition of that biological substance, notably on a MALDI-TOF type mass spectrometry analysis plate.

These objectives, among others, are achieved by the present invention, which firstly concerns an end fitting capable of being fitted to the body of a manual or automated device for sampling a biological substance of microbial origin, comprising:

a) a distal end comprising a means for sampling a biological substance of microbial origin,

b) a proximal free end intended to come into contact with the body of said sampling device and to enable the attachment of said end fitting to said body,

said end fitting being characterized in that all or some of the free distal end consists of a fibrous material having a porosity at least equal to 30%.

By a biological substance of microbial origin is essentially meant a biological substance consisting of bacteria, yeast or mold.

In one advantageous embodiment, the end fitting according to the invention consists entirely of a fibrous material having a porosity at least equal to 30%.

The fibrous material preferably has a porosity greater than 50%, preferably greater than 70%.

The fibrous material is advantageously selected from the group comprising: polyethylene, polyesters, polyethylene terephthalate (PET), PET/polyethylene copolymer, PET/PET copolymer, polyamide, cotton.

The sampling end fitting has a substantially conical or frustoconical overall shape. For its part, the sampling means is advantageously of cylindrical, frustoconical or spherical overall shape.

Another object of the present invention concerns a device for sampling a biological substance of microbial origin comprising:

an end fitting according to the invention

a body including at least:

a proximal part serving at least partially as a zone for gripping said device, and

a distal part having a free end to the end of which said end fitting is attached.

This device further includes a system for ejecting the end fitting. The ejection system advantageously includes a rod positioned inside said body and mobile in translation, so as to press on the end fitting and thus to eject it.

In one particular embodiment, said rod is mobile in translation by means of a pushbutton.

Another object of the invention concerns a process for sampling a biological substance of microbial origin comprising the following steps:

a) Positioning an end fitting according to the invention on the distal part of the sampling device,

b) Positioning the sampling device in the vicinity of a colony of a biological substance of microbial origin present on a culture medium in order to bring the sampling means of said end fitting into contact with the biological substance,

c) Sampling all or some of the biological substance with the aid of the sampling device, so that the sampled biological substance is attached to the sampling means.

Another object of the present invention concerns a process for preparing an analysis plate for MALDI-TOF type mass spectrometry microbiological analysis from a sample of a biological substance, comprising the following steps:

a) Positioning an end fitting on the distal part of the sampling device according to the invention,

b) Positioning the sampling device in the vicinity of a colony of a biological substance of microbial origin present on a culture medium in order to bring the sampling means of said end fitting into contact with the biological substance,

c) Sampling all or some of the biological substance with the aid of the sampling device, so that the sampled biological substance is attached to the sampling means,

d) Uniformly depositing the sampled biological substance on at least one analysis zone of a MALDI-TOF type mass spectrometry analysis plate, by bringing the sampling means into contact with the surface of said at least one analysis zone.

By microbiological analysis is essentially meant any analysis enabling identification of a microorganism, such as a bacterium or a yeast, but also enabling highlighting of any antimicrobial resistance marker, any characteristic of typing or of expression by said microorganism of a virulence factor.

Another object of the invention concerns a process for isolating a biological substance of microbial origin on a gelose culture medium, comprising the following steps:

a) obtaining a sample of a biological substance in contact with the sampling means of the end fitting of a sampling device according to the invention,

b) Positioning the sampling device in the vicinity of the surface of the gelose culture medium, so that the sampling means is in contact with said surface,

c) Moving the sampling device so that the sampling means is moved over the surface of the culture medium, while remaining in contact therewith, thereby releasing all or some of the sample of a biological substance in contact with said sampling means onto said surface of the culture medium.

In one particular embodiment, all of the processes described supra further include a final step of ejecting the sampling end fitting.

The sample of a biological substance may be obtained form a colony of a biological substance, by the sampling process described supra.

Alternatively, the sample of a biological substance may be obtained from a suspension of a biological substance. A suspension of this kind is traditionally obtained by putting one or more colonies of a biological substance into suspension in a saline solution. In accordance with this alternative, the sampling end fitting is dipped into a fraction of the suspension, in order to enable absorption of the liquid by the sampling means, thanks to the absorbing power of the fibrous material constituting said sampling means. The biological substance is then in contact with said sampling means. The concentration of the bacterial suspension is determined by the person skilled in the art according to the growth characteristics of the microorganism concerned. This forms part of their general background knowledge. Likewise, the suspension fraction used to charge the end fitting with biological substance is also determined deliberately. It is advantageously from a few microliters to a few tens of microliters.

The aims and advantages of the present invention will be better understood in the light of the following detailed and nonlimiting description of the invention given with reference to the drawings in which:

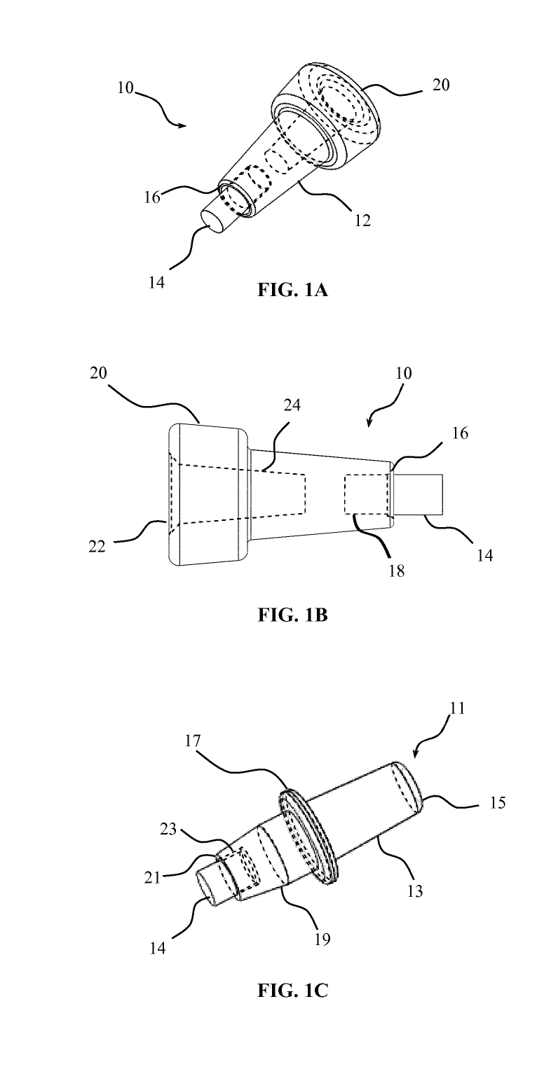

FIG. 1A represents a perspective view of a sampling end fitting according to a first embodiment of the present invention.

FIG. 1B represents a side view of the end fitting represented in FIG. 1A.

FIG. 1C represents a perspective view of a sampling end fitting according to a second embodiment.

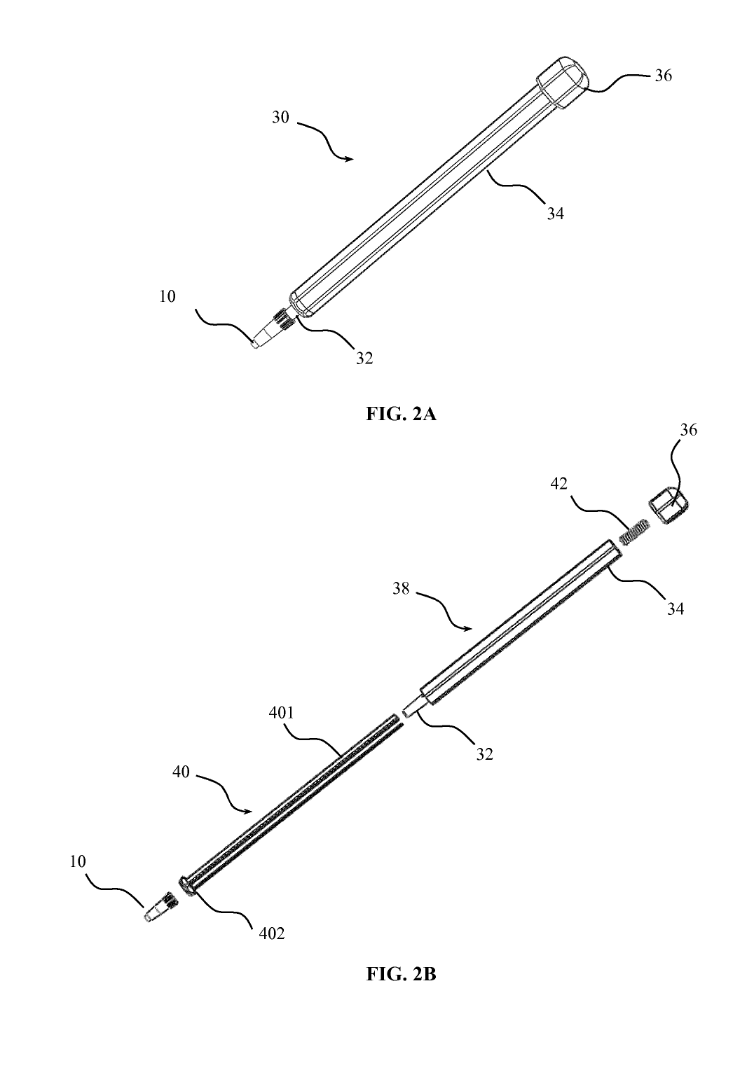

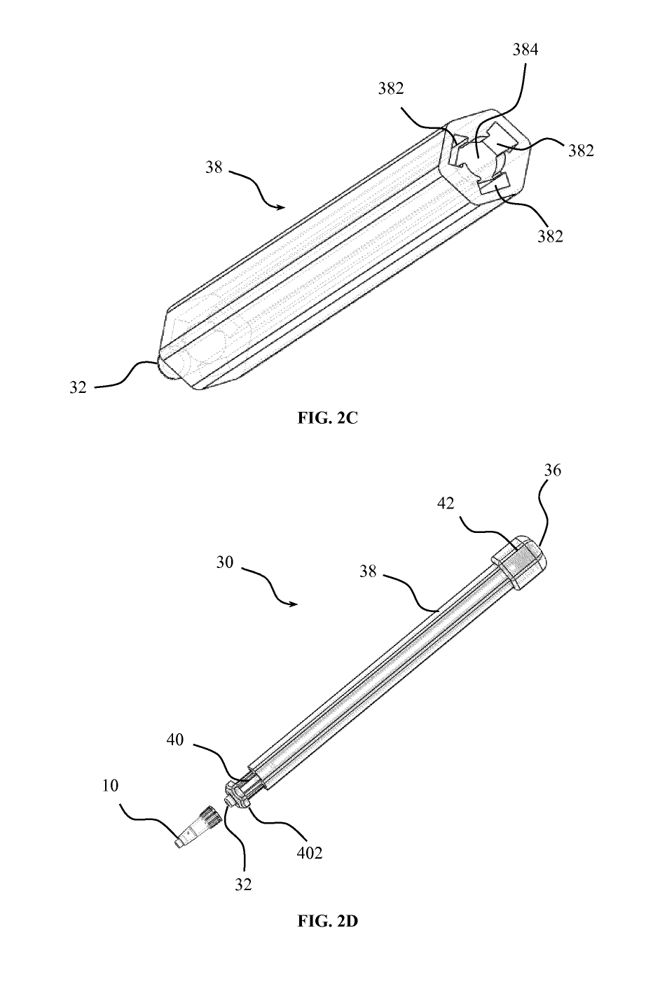

La FIG. 2A represents a side view of a biological substance sampling device in accordance with a first embodiment.

FIG. 2B represents an exploded side view of the biological substance sampling device represented in FIG. 2A.

FIG. 2C represents a perspective view of the body of the biological substance sampling device as represented in FIG. 2A.

FIG. 2D represents a side view of a biological substance sampling device as represented in FIG. 2A, in its configuration for ejecting the sampling end fitting.

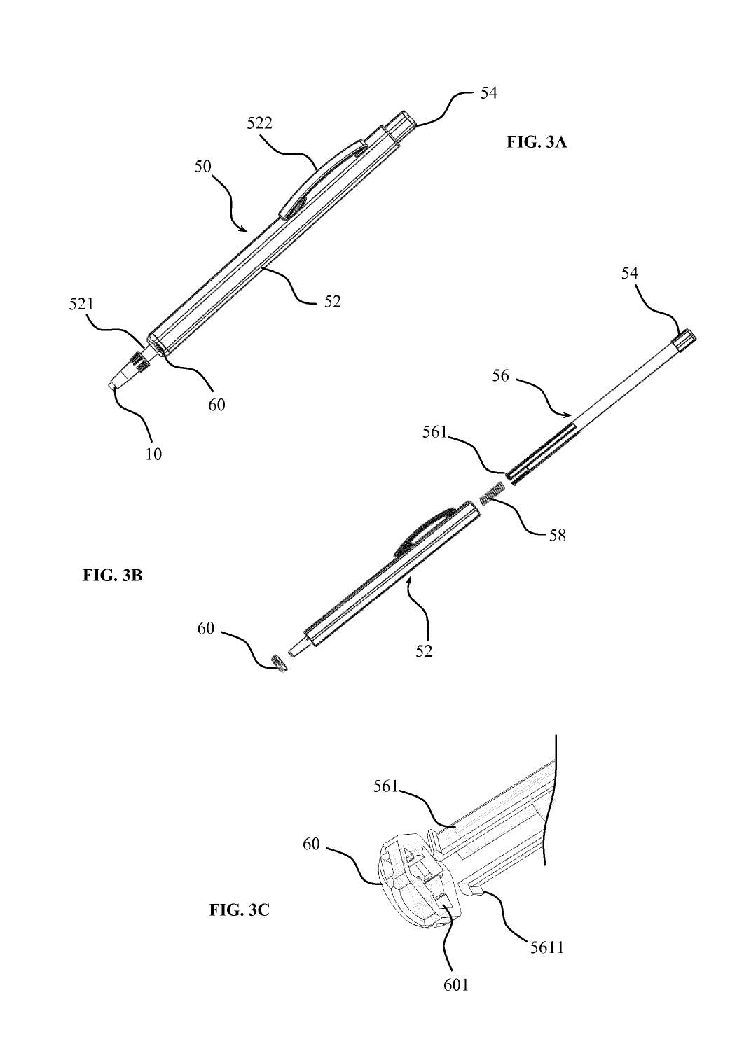

FIG. 3A represents a side view of a biological substance sampling device according to a second embodiment.

FIG. 3B represents an exploded side view of the biological substance sampling device represented in FIG. 3A.

FIG. 3C represents an exploded perspective view to a larger scale of the system for fixing the pusher-ring onto the rod of the sampling device represented in FIG. 3A.

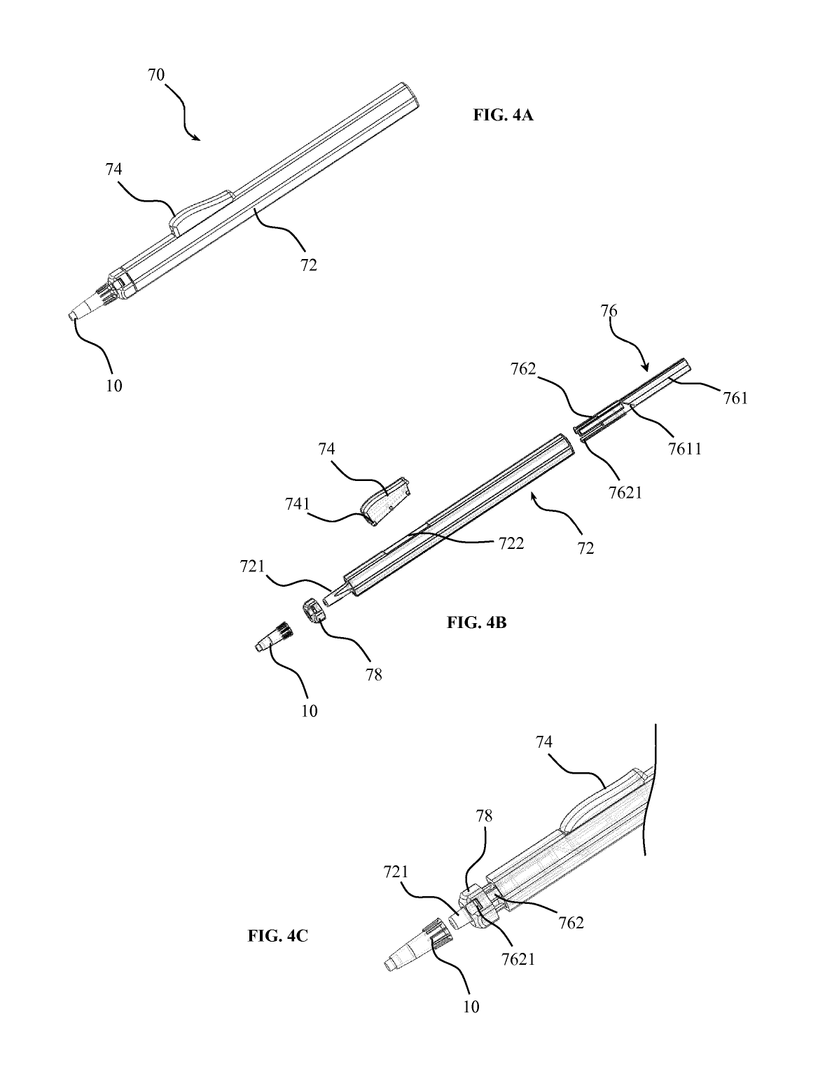

FIG. 4A represents a side view of a biological substance sampling device according to a third embodiment.

FIG. 4B represents an exploded side view of the biological substance sampling device represented in FIG. 4A.

FIG. 4C represents a view to a larger scale of the mechanism for ejecting the end fitting of the sampling device represented in FIG. 4A.

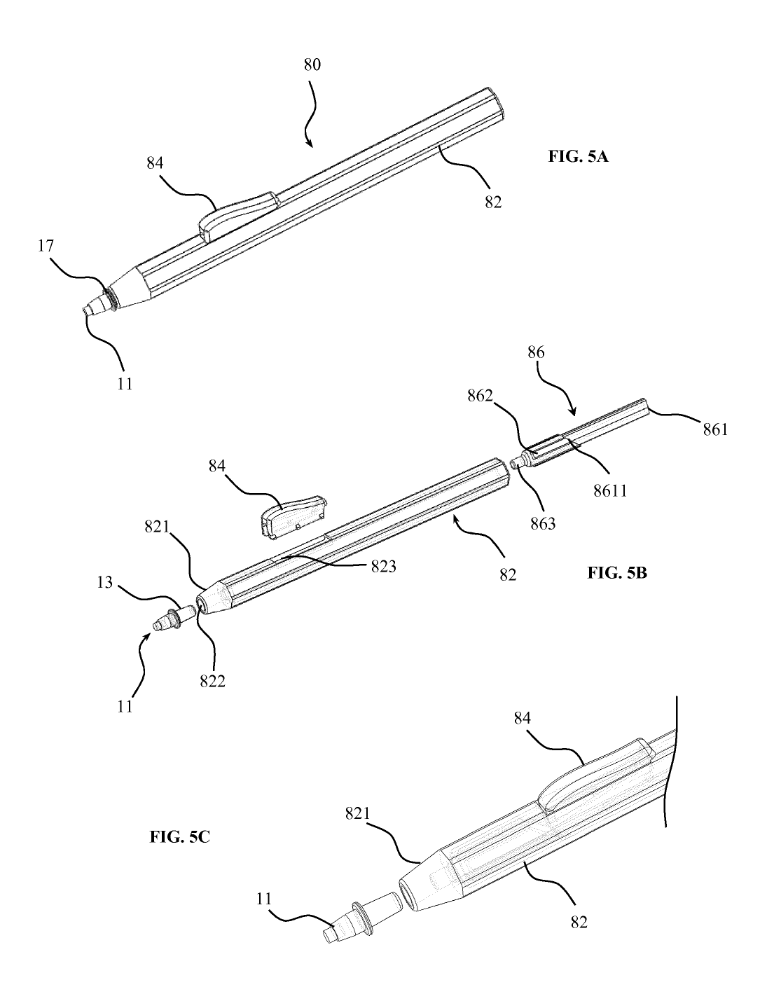

FIG. 5A represents a perspective view of a biological substance sampling device according to a fourth embodiment.

FIG. 5B represents an exploded perspective view of the biological substance sampling device represented in FIG. 5A.

FIG. 5C represents a perspective view of the biological substance sampling device represented in FIG. 5A in a configuration for ejecting the sampling end fitting.

In FIG. 1A, the end fitting 10 according to a first embodiment is represented in a perspective view. It is represented in side view in FIG. 1B. According to this embodiment, the end fitting 10 is of frustoconical general shape. It is nevertheless entirely feasible for the end fitting 10 according to the invention to be a different shape. It is constituted of three separate parts. Firstly, a distal part 12 of substantially frustoconical shape. This distal part 12 constitutes the part receiving a sampling means 14. To this end, the distal part 12 has a free end 16 at which is formed a blind cavity 18 in which the sampling means 14 is positioned. To this end, the shape of the cavity 18 and that of the sampling means 14 must be complementary. Ideally, the dimensions of the sampling means 14 must be slightly less than those of the cavity 18 to enable its insertion. However, it is preferable for them to be relatively close to prevent the sampling means escaping from the cavity. The end fitting 10 further includes a proximal part 20, also of frustoconical shape but shorter and wider than the distal part 12. The end 22 of the proximal part 20 is a free end and includes a blind cavity 24 in which the distal end of a sampling device comes to be housed.

The end fitting 10 may be molded from the materials usually employed for molding pipette end fittings. The material may for example be a polymer of polyolefin type. This type of material is generally inexpensive, can be sterilized and is suitable for use for the production of single-use products.

Regarding the sampling means 14, the material constituting the latter is very important. In fact, the sampling means has the two-fold technical constraint of having to provide for sampling a biological substance, such as a colony of bacteria, but also releasing said biological substance when it is deposited on an analysis device such as a mass spectrometry analysis plate. Thus the inventors discovered that the porosity of the material used to manufacture the sampling means 14 was an essential feature to enable a good compromise to be achieved between performance in terms of sampling and releasing the biological substance.

Moreover, over and above the inherent features of the sampling means, it is important to keep in mind that when the biological substance to be sampled is a colony of bacteria, how the latter behaves during sampling or during deposition can vary from one bacterial space to another. It is in fact well known that colonies of bacteria are more or less consistent, more or less viscous, more or less thread-like depending on the bacterial space concerned. It is therefore essential to have a sampling means that is able to manipulate any type of colony of bacteria, whatever its properties.

Thus the inventors established that a porosity at least equal to 30% was necessary to obtain the required properties. Ideally a porosity at least equal to 70% enables the best results to be obtained.

By porosity of the material is meant all of the voids (pores) of a solid material that can be filled with a fluid (liquid or gas). We also mean by porosity the physical parameter defined as the ratio between the volume of the voids and the total volume of the porous medium concerned.

The porosity of the material is measured in the following manner:

A sample of the dry fibrous material is taken.

The sample is weighed a first time.

The sample is immersed in water long enough to impregnate it completely.

The impregnated sample is weighed again.

The mass of water trapped in the material is deduced by calculating the difference between the mass of the impregnated material and that of the dry material.

The mass per unit volume of water being close to 1, there is deduced from this the volume of water trapped and thus the volume of the voids in the material.

The volume of the impregnated sample is then measured.

By dividing the value for the voids in the sample of material obtained above via the measured volume of the impregnated sample it is therefore possible to obtain the value of the porosity of the material.

The materials particularly suitable for producing the sampling means may be fibrous materials. Among these materials may be cited synthetic materials such as polyethylenes, polyesters, polyamides. Among the polyesters may be mentioned polyethylene terephthalate (PET). They may also be copolymers, such as polyethylene/polyester polymers, such as a polyethylene/PET copolymer or again a PET/PET copolymer. When it is a question of a fibrous material, the fibers may consist of a mono-component or bi-component material. A bi-component fiber may for example consist of a PET core and a polyethylene sheath.

Natural fibrous materials may equally be used. This is the case of cotton fibers in particular.

A second embodiment of the end fitting is represented in FIG. 1C. This end fitting 11 consists of three parts: a substantially cylindrical or possibly frustoconical proximal part 13. In contrast to the first embodiment, the free end 15 of the proximal part 13 has no blind cavity and is plugged. In fact, according to this embodiment, the distal end of the sampling device does not come to be inserted in the end fitting 11. It is in contrast the end fitting 11 that comes to be inserted in the distal end of the sampling device, and to be more precise its proximal end 15. Of course, to do this, the sampling device must have a free distal end able to receive the end fitting 11. It must therefore include a cavity to receive the end fitting 11 the dimensions of which must be slightly greater than the dimensions of the proximal part 13. The end fitting 11 includes a flange 17 that enables ejection of said end fitting after use. It can also serve as an abutment when the end fitting 11 is inserted in the sampling device. Finally, the end fitting 11 includes a distal part 19 intended to receive a sampling means 14, as in the case of the end fitting 10 according to the first embodiment. To this end, the distal part 19 has a free end 21 in which is formed a blind cavity 23 in which the sampling means 14 is positioned.

According to a variant embodiment of the sampling end fitting 11, the proximal end 13 may include an orifice enabling a hole to be generated passing between the distal and proximal ends. It is then possible to use a longer sampling means, positioned in the through-hole and fastened to the end fitting at the level of the orifice at the proximal end, or even at both ends. A variant of this kind is of simpler design and therefore less costly to manufacture.

The sampling means 14 represented in FIGS. 1A to 1C is of cylindrical shape. It is nevertheless possible for the sampling means 14 to be a different shape. Accordingly, the free end of the latter may for example be conical to improve further the accuracy of sampling and of depositing the biological substance. The free end of the end fitting 14 may also have any other shape suitable for sampling a biological substance.

According to one variant of the invention, a sampling end fitting consisting entirely of porous material could be envisaged. According to this variant embodiment, the sampling end fitting would also constitute the sampling means.

According to another variant of the invention, the sampling means, the sampling end fitting and the sampling device could be one and the same. In fact, a single-use sampling device could be envisaged to which a porous sampling end fitting is fastened. Alternatively, the sampling device may be made entirely of porous material. To this end, the dimensions of the sampling means must allow easy gripping and manipulation.

The sampling end fitting conjugate with the sampling means or the sampling means alone, if it is in one piece with the sampling end fitting, may be adapted to be used in an automated system for sampling a biological substance. A system of this kind could for example sample automatically colonies of bacteria on Petri dishes and prepare mass spectrometry analysis plates.

In FIG. 2A is represented a sampling device 30 according to a first embodiment. This sampling device 30 includes a distal part 32, an intermediate part 34 and a proximal part 36. The function of the substantially frustoconical distal part 32 is to carry the sampling end fitting 10 according to the invention. The function of the intermediate part 34, also cylindrical, is to allow gripping of the sampling device 30 by its user. Finally, the proximal part 36 has the general shape of a bucket, the inside diameter of which is slightly greater than the outside diameter of the intermediate part 34, so that the proximal part can partially cover the proximal end of the intermediate part.

FIG. 2B represents the sampling device 30 in an exploded view in which each component of this device is represented separately. It is therefore seen that the distal part 32 and the intermediate part 34 are in fact one and the same component 38 constituting the body of the sampling device.

As represented in FIG. 2C, this substantially cylindrical body 38 has longitudinal and peripheral openings 382 through it together with a central opening 384. The openings 382 are arranged so that each receives a blade 401 of the rod 40, as represented in FIG. 2B. As for the opening 384, it is adapted to receive a coil spring 42, as represented in FIG. 2B. The rod 40 is therefore mainly constituted of three blades 401 the base of which is fastened to a ring-abutment 402. The rod 40 comes to be positioned inside the body of the device 38 on introducing the free ends of the blades 401 into the openings 382 until the ring-abutment 402 comes to bear against the distal end of the intermediate part 34, while the distal part 32 comes to pass through the opening of said ring-abutment 402. At the opposite end of the body 38 the lower end of the coil spring 42 comes to be housed in the opening 384 while its upper end is placed in contact with the interior face of the proximal part 36 of the sampling device, which comes to cap the body 38. This proximal part serves as a pusher member on ejecting the sampling end fitting 10 when the latter has been used.

The blades 401 are longer than the intermediate part 34 so that when the rod 40 is positioned inside the body 38 the free top ends of the blades 401 emerge from the openings 382. These free ends are fastened to the pusher member 36 when the spring is in place. This fastening may be mechanical. Accordingly, placing radially projecting lugs in the vicinity of the free end of the blades 401 that come to be housed in openings formed in the internal wall of the pusher member 36 may be envisaged. Alternatively, it is possible to fasten the free ends of the blades 401 to the internal face of the pusher member 36 by chemical bonding.

As represented in FIG. 2D, when pressure is exerted on the pusher member 36 along the longitudinal axis of the sampling device, the latter member slides on the body 38. This sliding action is communicated to the rod 40 that is fastened to the pusher member 36 and slides inside the body 38. This sliding is manifested at the distal end of the sampling device by movement in translation of the ring-pusher member 402. As the latter bears against the proximal end of the end fitting 10, it drives said end fitting in movement in translation so that it is detached from the distal end 32 of the sampling device 30. The pressure exerted on the coil spring 42 by the pusher member 36 leads to compression thereof. This compression is transmitted to the rod 40, which slides as explained supra. When the pressure on the proximal part 36 is released, the spring 42 tends to return to a rest position in which it no longer presses on the rod 40. The latter then resumes its initial position by reverse movement in translation, freeing the distal part 32 which is then available to receive another sampling end fitting.

The various steps of the process for using the sampling device 30 with a sampling end fitting 10 may therefore be summarized as follows:

An end fitting 10 is positioned on the free end 32 of the sampling device. This step is generally carried out by positioning the device vertically in line with a sterile end fitting, generally positioned on a suitable support. The end of the distal part 32 of the sampling device is then inserted into the cavity 24 of the proximal part 20 of the end fitting 10 until it abuts on the bottom of the cavity 24.

To sample a biological substance such as colony of microorganisms on a gelose culture medium, the sampling device is positioned so that the sampling means of the end fitting comes into contact with the colony of microorganisms. All or part of the colony is then sampled.

To deposit all or part of the sampled biological substance on a surface such as the surface of a MALDI-TOF mass spectrometry analysis plate, the sampling device is moved toward said surface, so that the end of the sampling means carrying the biological substance comes into contact with the surface. Small circular movements are then performed with the sampling device to deposit a layer of the biological substance on said surface.

Once the deposit has been applied, the sampling end fitting is ejected by actuating the ejection means and thrown into a bin. In the case of the sampling device as represented in FIGS. 3 to 5, ejection is effected by exerting pressure on the proximal part 36.

A second embodiment of the sampling device is represented in FIGS. 3A to 3C. This sampling device 50 consists of a substantially cylindrical hollow body 52. This body 52 has in its distal part a free end 521 of substantially frustoconical shape, intended to carry a sampling end fitting 10. This free end 521 is fastened to the body 52 by three fins (not shown) defining three interstitial spaces. The body 52 also includes an attachment system or clip 522 enabling the sampling device to be attached to the pocket of a garment, for example, like a pen. Finally, in FIG. 3A, there is also seen a pushbutton 54 which exits the proximal end of the body 52. As can be seen in FIG. 3B, the pushbutton 54 is fastened to a rod 56 placed inside the body 52. This substantially cylindrical rod 56 ends at its terminal end in three attachment lugs 561 represented in close up in the larger scale view 3C. The central space between said attachment lugs 561 is intended to receive a coil spring 58, as shown in FIG. 3B.

The rod 56 is inserted in the body 52 via the proximal end of the latter, the coil spring 58 being inserted first. Said coil spring 58 comes to be positioned so that it bears against the fins and the upper part of the distal end 521. Once inserted, the rod 56 comes to be positioned so that it straddles the spring so that the attachment lugs 561 come to cross the interstitial spaces defined between the fins and emerge at the base of the body 52 around the free end 521. A ring 60 is fitted around the free end 521. This ring 60 has three openings 601 intended to receive the end of the attachment lugs 561. The attachment lugs have at their end radial tabs 5611 which once the lugs are positioned inside the ring 60 will come to be locked into the openings 601 and thus fasten together the ring 60 and the rod 56. Alternatively, and with the aim of simplifying the mechanism, it is entirely possible to replace this mechanical fastening system with a system including lugs with no tabs stuck to the ring. As represented in FIG. 3A, the ring 60 comes to bear against the body 52 at the level of the upper part of the free end 521 because of the action of the coil spring 58 which comes to exert on the rod 56 a repulsion force toward the upper part of the body 52.

As in the first embodiment of the sampling device 30, when pressure along the longitudinal axis of the sampling device is exerted on the pushbutton 54, this pressure is transmitted to the rod 56 fastened to the pushbutton 54. The latter then comes to slide inside the body 52, thus compressing the coil spring 58. This sliding is manifested at the distal end of the sampling device by movement in translation of the ring 60, so that the latter comes to bear on the proximal end of the end fitting 10 and to drive the latter in translation until it is detached from the distal end 521 of the sampling device 50. When the pressure exerted on the coil spring 58 by the rod 56 is released, because the pressure on the pushbutton 54 is released, the latter spring resumes its rest position. The rod 56 is driven in the reverse translation direction by the pressure of the spring until the ring 60 comes to bear against the body 52, rendering the distal part 521 accessible again to receive another sampling end fitting 10.

A third embodiment of a sampling device is represented in FIGS. 4A to 4C. This sampling device 70 consists of a substantially cylindrical hollow body 72. This body 72 has in its distal part a free end 721 of substantially frustoconical shape, intended to carry a sampling end fitting 10. This free end 721 is fastened to the body 72 by three fins (not shown) defining three interstitial spaces. Also seen is a pushbutton 74 positioned laterally on the body 72, which comes to be inserted in an opening 722 formed in the lateral wall of the body 72, as can be seen in FIG. 4B. It also includes a rod 76 that is inserted in the body 72 at its upper end. This rod 76 consists of a one-piece proximal part 761 of substantially rectangular cross section and a distal part consisting of three attachment lugs 762. At the interface with the distal part, the proximal part 761 has on these two opposite faces an inclined shoulder 7611, intended to come to cooperate with the pushbutton 74. The rod 76 is inserted in the body 72 of the sampling device 70, via the proximal end of the latter. Once the rod 76 has been inserted, the attachment lugs 762 come to cross the interstitial spaces defined between the fins retaining the free end 721 and to exit at the base of the body 72 around said free end 721. A ring 78 is inserted around the free end 721. This ring 78 is similar to the ring 60 described supra and includes three openings intended to receive the ends of the attachment lugs 762, also provided with radial tabs 7621 and intended to cooperate with the openings in the ring 78 to fasten the rod 76 to the ring 78, as shown in FIG. 4C.

The pushbutton 74 has an inverted “U” shape cross section, intended to enable it to be positioned around and straddling the proximal part 761 of the rod 76, particularly at the level of the inclined shoulders 7611. The pushbutton 74 has on each of these two lateral faces three lugs 741 intended to prevent it escaping, once position in the housing 722. When the rod 76 and the pushbutton 74 are positioned in the body 72 of the sampling device 70, the pushbutton 74 comes to bear at the level of its distal part against the inclined shoulders 7611, preventing the rod 76 from escaping. The interaction between the pushbutton and the inclined shoulders 7611 enables movement of the rod 76 between a rest position and a position for ejecting the sampling end fitting 10. In fact, when an end fitting 10 is positioned on the distal end 721 of the sampling device 70, the latter comes to bear against the ring 78. The latter is then positioned at a high position, against the body 72, as shown in FIG. 4A. The rod is then in a rest position, in which the pushbutton 74 is again in the high position.

When the user of the sampling device 70 wishes to eject the sampling end fitting 10, they exert pressure on the pushbutton 74. This pressure leads to the pushbutton being depressed in the housing 722, which then slides on the inclined shoulders 7611. The pushbutton being unable to move laterally, its sliding on the inclined shoulders 7611 leads to movement of the rod 76 in horizontal translation toward the distal end of the body 72. The rod then goes from its rest position to its position for ejecting the end fitting, in which the ejection ring 78 is again in a low position after it has exerted pressure on the end fitting, and this leads to ejection of the latter, as represented in FIG. 4C.

A fourth embodiment is represented in FIGS. 5A to 5C. This embodiment is very similar to the third embodiment where the operation of the sampling device is concerned. In fact, the sampling device 80 represented in FIGS. 5A to 5C consists of a substantially cylindrical hollow body 82. This body 82 is in one piece and has in its distal part a free end 821 of substantially frustoconical shape. This end 821 is pierced by a circular orifice 822. This orifice is intended to receive the proximal part 13 of a sampling end fitting 11, as described with reference to FIG. 1C. In this configuration, the sampling end fitting 11 is depressed in the body of the sampling device 80 until the flange 17 of the end fitting 11 comes to bear against the end 821 of the body 82. The body 82 also includes a pushbutton 84 positioned laterally on the body 82, which comes to be inserted in a housing 823 formed in the lateral wall of the body 82. It finally includes a rod 86 which is inserted in the body 82 by its upper end. This rod 86 consists of a one-piece proximal part 861 of substantially rectangular cross section, a one-piece distal part 862 of substantially round cross section ending in a nipple 863. At the interface between the proximal part 861 and the distal part 862 two inclined shoulders 8611 are formed on respective opposite sides of the proximal part 861. These shoulders 8611 and the pushbutton 84 are caused to cooperate via a mechanism identical to that described supra with reference to the sampling device 70 represented in FIGS. 4A to 4C, so that when the user presses on the pushbutton 84, they trigger the movement in translation of the rod 86 in the opening in the body 82, toward the distal end of the latter. The nipple 863 then comes to bear against the free end 15 of the proximal part 13 of the end fitting 11 and exerts a pressure on the latter so that the end fitting 11 is ejected from the sampling device 80 by movement in translation. This is clearly shown in FIG. 5C. When positioning a new fitting 11 on the sampling device 80, the proximal part 13 that penetrates into the opening in the body 82 via the orifice 822 comes to push the rod 86 in the opposite direction, leading to the pushbutton 84 rising by movement in vertical translation thanks to the inclined shoulders 8611 that cooperate with the pushbutton 84.

Other embodiments of the sampling device can be envisaged. Accordingly, one possible other embodiment consists in positioning at the distal end but on the body at its external part a mechanical system intended to come to bear against the end fitting to allow its ejection. For example there a sleeve mobile in translation on the body of the sampling device may be in contact with the sampling end fitting and enables the latter to be ejected when it is moved by the user.

Other protocols for preparation of samples of a biological substance with a view to analysis using the sampling device may be envisaged. Accordingly, once the biological substance has been sampled, it is possible to prepare a suspension of the biological substance in an ad hoc solution, such as a saline solution. To do this, releasing the sampling end fitting carrying the biological substance directly into a tube containing a quantity of solution in suspension may be envisaged. Vortex agitation of the tube containing the end fitting will then enable release of the biological substance retained on the fibrous material.

The sampling device can also be used to carry out isolation on gelose culture media. This isolation may be carried out directly from colonies sampled with the sampling device according to the invention and then directly reseeded on a gelose culture medium. It can equally be produced from suspensions of a biological substance, produced with the aid of the sampling device, as described supra.

When the sampling device is used to carry out isolation, it is preferable for the sampling means to have a spherical end and to be of sufficient diameter at its end to prevent pressing it against the gelose culture medium coming to damage the latter. A suitable diameter is for example between 2 and 7 millimeters (mm) and advantageously between 2 and 4 mm.

Isolation may be effected from a suspension of bacteria the concentration of which is, for example, 10⁷ CFU (Colony Forming Units)/mL (milliliter). With such a concentration, a sample of 5 microliters (μL) of suspension using the sampling means is sufficient to enable isolation of good quality on the gelose culture medium. The 5 μL of suspension is aspirated by the material constituting the sampling means. Said sampling means charged with suspension can then enable the progressive release of that suspension when it is applied to the gelose culture medium. This progressive release is allowed thanks to the absorbent nature of the material constituting the sampling means. The consequence of this is improved isolation quality.

The isolation may be done in accordance with the traditional techniques, such as the so-called “dial” technique or the so-called “spiral” technique.

### EXAMPLES

### Example 1: Use of a Sampling Device with Sampling End Fittings for the Preparation of a Plate for MALDI-TOF Analysis

The sampling device used in this example is the device represented in FIGS. 2A to 2D and described supra.

The sampling end fitting is made of plastic material, such as those conventionally used to produce disposable pipette end fittings. It includes a sampling means of cylindrical shape of 2 mm diameter and 6 mm length. It consists of fibers of polyethylene terephthalate (PET)/PET copolymer, a material marketed by the company Porex under the reference PSU-832.

The body of the sampling device is injection molded from polypropylene. It has a length of 120 mm and a section of hexagonal shape with 8.2 mm between the opposite faces of the hexagon, which allows easy gripping like a pencil, with no risk of muscular-skeletal problems.

The sampling device is used to sample colonies of different species of bacteria, which have grown on Columbia agar+5% sheep blood (COS cell line) culture medium, marketed by the applicant under the reference 43041.

The sampled colonies are deposited on a disposable MALDI-TOF analysis plate with 48 positions, marketed by the applicant under the reference 410893.

Of the species chosen, some are known to form colonies that are difficult to sample. This is the case for example with Bacillus lichenformis, Klebsiella pneumoniae, Proteus mirabilis or again Nocardia asteroides.

The process is carried out in accordance with the following steps:

1. A sampling end fitting is positioned on a sampling device.

2. A colony of bacteria is sampled on a COS cell line type culture medium, with the aid of the sampling device, by bringing the sampling means made of fibrous material carried by the sampling end fitting into contact with the colony.

3. The bacterial sample is deposited on the MALDI-TOF analysis plate, in one of the 48 positions provided for this purpose, by bringing the sampling means carrying the bacterial sample into contact with the MALDI-TOF plate, exerting pressure and spreading the sample. The pressure exerted on the fibrous material of the sampling means enables retention of surplus sample on said fibrous material. It is possible to carry out successive depositions at different positions on the analysis plate when it wished to replicate the analysis. These other deposits may be applied using the same end fitting.

4. When all the deposits have been applied, the sampling end fitting is ejected into a bin by exerting pressure on the pushbutton, as described supra.

5. 1 μL of matrix (alpha cyano 4-hydroxycinnamic acid ready for use, bioMerieux reference 411071) is deposited on each deposit of the biological substance, with the aid of a micropipette.

6. Steps 1 to 5 are repeated for each colony that it is wished to analyze,

7. An E. coli ATCC 8739 strain used for calibration and as a positive control is also deposited in accordance with steps 1 to 5.

8. The analysis plate is stored for the time necessary for the matrix to dry.

9. When the plate is entirely dry, it is introduced into the MALDI-TOF VITEK® MS mass spectrometer marketed by the applicant and the mass spectra are acquired.

10. The microorganisms are identified by spectrum analysis using the expert system and the VITEK® MS database.

The same colonies of bacteria are analyzed using the traditional process for MALDI-TOF analysis plate preparation employing an oese and well known to the person skilled in the art.

The results obtained with the two analysis plate preparation methods are set out in TABLE 1 and compared.

The symbol “✓” signifies that the spectrum is of good quality and enables the expected identification of the species.

The symbol “X” means that the spectrum is not of sufficient quality to enable identification of the species.

TABLE 1

| | | Protocol using |
|---|---|---|
| | Reference protocol | the device according |
| Species | using an oese | to the invention |
| B. lichenformis | X | ✓ |
| (strain API 0608016) | | |
| E. aerogenes | ✓ | ✓ |
| (strain ATCC 13048) | | |
| E. coli | X | ✓ |
| (strain ATCC 25922) | | |
| E. faecalis | ✓ | ✓ |
| (strain API 1006023) | | |
| K. oxytoca | X | ✓ |
| (strain API 1006024) | | |
| K. pneumoniae | X | ✓ |
| (strain ATCC 13883) | | |
| K. pneumoniae ssp ozaenae | X | ✓ |
| (strain API 1006025) | | |
| N. asteroides | ✓ | ✓ |
| (strain API 1201096) | | |
| N. farcinica | X | ✓ |
| (strain API 1201094) | | |
| P aeruginosa | ✓ | ✓ |
| (strain ATCC 27853) | | |
| P. mirabilis | X | ✓ |
| (strain ATCC 12453) | | |
| S. aureus | ✓ | ✓ |
| (strain ATCC 29213) | | |
| S. epidermidis | ✓ | ✓ |
| (strain ATCC 12228) | | |
| Time necessary for sampling | 16 minutes | 12 minutes |
| and depositing samples | | |

It follows from the analysis of Table 1 that, using the protocol employing the sampling device according to the invention, all the species analyzed are correctly identified by mass spectrometry using the VITEK® MS.

By comparison, with the traditional protocol using an oese to sample colonies of bacteria, only 6 species of 13 analyzed are correctly identified. Of the species not identified, a majority are those known to form colonies difficult to manipulate.

In a particularly advantageous manner, the process for the invention therefore enables sampling and depositing of bacteria species forming colonies with an atypical consistency. Accordingly, it is possible easily to obtain excellent identification by MALDI-TOF mass spectrometry results powdery and crust-forming species such as B. lichenformis or N. farcinica, adherent species such as K. pneumoniae and species that diffuse on gelose such as P. mirabilis.

It is also found that the time necessary to deposit 48 samples is shorter with the sampling device according to the invention (12 min) than with an oese (16 min).

The process for the present invention is therefore more ergonomic, easier to use, faster and enables better results to be obtained than the prior art reference process.

### Example 2: Comparison of the Performance in Identification of Different Species of Bacteria by Mass Spectrometry Analysis Using Different Fibrous Materials

In this example, a plurality of fibrous materials having different porosities were tested, notably for their capacity to enable effective sampling of biological substances coming from colonies of different species of bacteria, but equally in their ability to produce a homogeneous deposit on a MALDI-TOF analysis plate. In fact, the performance in identification of species of bacteria by MALDI-TOF mass spectrometry is directly linked to the quality of the deposit of the biological substance on the analysis plate whatever the quantity of material or the homogeneity of the deposit.

The various species were therefore subjected to MALDI-TOF mass spectrometry analysis in order to identify them.

The analysis protocol is that described for example 1, including the sampling device.

The materials tested are set out in TABLE 2 below:

TABLE 2

| | Diameter | Length | Dry weight | Weight (mg) | V1 | V2 | |
|---|---|---|---|---|---|---|---|
| | mm | mm | mg | water + material | air mm³ | tot mm³ | Porosity |
| Cotton | 28 | 36 | 2412 | 24160 | 21748 | 22156 | 98% |
| PET/PET | 2.5 | 5.9 | 6.1 | 27 | 21 | 29 | 74% |
| copolymer | | | | | | | |
| PSU892 | | | | | | | |
| XA-20521-PS | 2.1 | 13.4 | 33.9 | 52 | 18 | 46 | 39% |
| PE | | | | | | | |

In TABLE 2 are indicated the dimensions of the samples of materials used. Moreover, the values enabling calculation of the porosity by the method indicated supra have also been indicated.

The material PSU892 is a material obtained from two-component fibers, consisting of a polyethylene terephthalate envelope and a polyester core. A material of this kind is marketed by the company POREX®. It is moreover described in the patent application WO-A-03/080904.

The material XA-20521-PS is a material based on polyethylene. A material of this kind is marketed by the company POREX® under the trade name NIBS.

In order to carry out the tests, three different species of bacteria were used:
Staphylococcus epidermidis (strain ATCC 12228)
Klebsiella pneumoniae (strain ATCC 13883)
Escherichia coli (strain ATCC 25922)

These species were each seeded on a Columbia gelose culture medium containing 5% of sheep blood (COS), marketed by the applicant under the reference 43041.

For each species, a colony is sampled with the sampling device according to the invention and four deposits are made at four contiguous positions of the MALDI-TOF analysis plate.

The results are set out in TABLE 3 below:

TABLE 3

| | | Analysis | Identification |
|---|---|---|---|
| Material | Species | result | percentage |
| PSU 892 | Staphylococcus | 100 | 100% |
| | Aureus | 100 | |
| | | 100 | |
| | | 100 | |
| | Klebsiella | 99.99 | |
| | pneumoniae | 99.99 | |
| | | 99.99 | |
| | | 99.99 | |
| | Escherichia coli | 99.99 | |
| | | 99.99 | |
| | | 89.43 | |
| | | 95.14 | |
| Cotton | Staphylococcus | 99.99 | 100% |
| | Aureus | 99.99 | |
| | | 99.99 | |
| | | 99.99 | |
| | Klebsiella | 99.99 | |
| | pneumoniae | 99.99 | |
| | | 99.99 | |
| | | 99.99 | |
| | Escherichia coli | 99.48 | |
| | | 98.41 | |
| | | 99.76 | |
| | | 99.91 | 83.33% |
| XA- | Staphylococcus | 99.99 | |
| 20521- | Aureus | 99.99 | |
| PS | | 99.99 | |
| | | 99.99 | |
| | Klebsiella | 99.99 | |
| | pneumoniae | 99.99 | |
| | | 99.99 | |
| | | 99.99 | |
| | Escherichia coli | — | |
| | | 97.53 | |
| | | — | |
| | | 80.23 | |

It is seen that for the materials having a high porosity value (greater than 70%), identification performance is excellent, in that all of the species are correctly identified. This is the case with cotton or the material PSU 892.

On the other hand, when materials are used having lower porosity values, the identification performance is found to be impacted. Accordingly, with the material XA-20521-PS which has a porosity of 39%, it is seen that two of the four deposits of Escherichia coli did not allow the identification of this bacterium. Thus there results an identification rate of only 83.3%.

1. An end fitting capable of being fitted to the body of a manual or automated device for sampling a biological substance of microbial origin, comprising:
a) a distal end comprising a means for sampling a biological substance of microbial origin,
b) a proximal free end intended to come into contact with the body of said sampling device and to enable the attachment of said end fitting to said body,
said end fitting being characterized in that all or some of the free distal end consists of a fibrous material having a porosity at least equal to 30%.

2. The end fitting as claimed in claim 1, consisting entirely of a fibrous material having a porosity at least equal to 30%.

3. The end fitting as claimed in claim 1, in which the fibrous material has a porosity greater than 50%.

4. The end fitting as claimed in claim 1, in which the fibrous material is selected from the group comprising: polyesters, polyethylene, polyethylene terephthalate (PET), PET and polyethylene copolymer, PET/PET polyamide copolymer, cotton.

5. The end fitting as claimed in claim 1, having a substantially conical or frustoconical overall shape.

6. The end fitting as claimed in claim 1, in which the sampling means is of cylindrical, frustoconical or spherical overall shape.

7. A device for sampling a biological substance of microbial origin comprising:
an end fitting as claimed in claim 1
a body including at least:
a proximal part serving at least partially as a zone for gripping said device, and
a distal part having a free end to the end of which said end fitting is attached.

8. The device as claimed in claim 7, further comprising a system for ejecting the end fitting.

9. The device as claimed in claim 8, in which the ejection system includes a rod positioned inside said body and mobile in translation.

10. The device as claimed in claim 9, in which said rod is mobile in translation by means of a pushbutton.

11. A process for sampling a biological substance of microbial origin comprising the following steps:
Positioning the sampling device as claimed in claim 7 in the vicinity of a colony of the biological substance of microbial origin present on a culture medium in order to bring the sampling means of said end fitting into contact with the biological substance,
Sampling all or some of the biological substance with the aid of the sampling device, so that the sampled biological substance is attached to the sampling means.

12. A process for preparing an analysis plate for MALDI-TOF type mass spectrometry microbiological analysis from a sample of a biological substance, comprising the following steps:
Positioning the sampling device as claimed in claim 7 in the vicinity of a colony of a biological substance of microbial origin present on a culture medium in order to bring the sampling means of said end fitting into contact with the biological substance,
Sampling all or some of the biological substance with the aid of the sampling device, so that the sampled biological substance is attached to the sampling means,
Uniformly depositing the sampled biological substance on at least one analysis zone of a MALDI-TOF type mass spectrometry analysis plate, by bringing the sampling means into contact with the surface of said at least one analysis zone.

13. A process for isolating a biological substance of microbial origin on a gelose culture medium, comprising the following steps:
a. obtaining a sample of the biological substance in contact with the sampling means of the end fitting of a sampling device as claimed in claim 6,
b. Positioning the sampling device in the vicinity of the surface of the gelose culture medium, so that the sampling means is in contact with said surface,
c. Moving the sampling device so that the sampling means is moved over the surface of the culture medium, while remaining in contact therewith, thereby releasing all or some of the sample of the biological substance in contact with said sampling means onto said surface of the culture medium.

14. The preparation process as claimed in claim 11, further comprising a final step of ejecting the sampling end fitting.
